(19) United States
(12) Patent Application Publication
(10) Pub. No.: US 2011/0195525 A1
(43) Pub. Date: Aug. 11, 2011

(54) Method of use and Apparatus for an Enhanced Lateral Flow Rapid Test Device

(76) Inventor: Arnold J. AQUILINO, Encinitas, CA (US)

(21) Appl. No.: 13/025,555

(22) Filed: Feb. 11, 2011

Related U.S. Application Data
(60) Provisional application No. 61/303,418, filed on Feb. 11, 2010.

(51) Int. Cl. G01N 33/558; G01N 30/00; G01N 1/00

(52) U.S. Cl. 436/514; 422/69; 436/174

(57) ABSTRACT

An immunoassay test membrane strip that is able to quickly tell the experimenting user the results of the presence of a specific antibody. The present invention makes use of a membrane strip that is broken into different sections. The user applies a specimen and a dilution buffer to the membrane strip. The specimen and the labeled conjugate solution travel through the membrane strip through capillary action. The membrane strip will provide the user with visual results on the presence of the antibody that the specimen is being tested for and a visual result of the validity of the test.

The current application claims a priority to the U.S. Provisional Patent application Ser. No. 61/303,418 filed on Feb. 11, 2010.

### FIELD OF THE INVENTION

This invention relates generally to a device used for immunoassay testing. The present invention makes use of a lateral flow technique in determining the presence of an analyte.

### BACKGROUND OF THE INVENTION

Traditional Lateral flow assays use the form dipsticks where the sample is allowed to flow along a substrate through capillary action. During its flow the sample will encounter a coloring agent and continue along the substrate. The sample with the coloring agent will then encounter lines or zones which have been pretreated with antibodies or antigens. If a specific analyte being tested for is present in the sample, the sample with the coloring agent will collect in the lines or zones pretreated with the antibodies or antigens. The presented invention makes use of such this traditional technique and improves on it to decrease the time when results are shown and increase assay sensitivity. The present invention provides a membrane strip that comprises of a plurality of sections that allow the user to perform the immunoassay procedure. The user can simply apply a specimen and dilution buffer to the present invention to initiate the assay. The specimen and dilution buffer are able to laterally flow through the membrane strip by means of capillary action. The assay provided by the present invention is able to provide a result of the test by means of a colorimetric line.

### BRIEF DESCRIPTION OF THE DRAWINGS

FIG. 1 is a view of the present invention and the different sections used for the immunoassay procedure.

FIG. 2 is a view of the antibodies from the specimen being captured by the antigen on the specific antigen capture line.

FIG. 3 is a view of the conjugated labels being solubilized by the dilution buffer then binding to the antibody-antigen complexes on the specific antigen capture line. The conjugated labels provide the specific antigen capture line with a visible colorimetric line.

### DETAIL DESCRIPTIONS OF THE INVENTION

All illustrations of the drawings are for the purpose of describing selected versions of the present invention and are not intended to limit the scope of the present invention.

The present invention is a membrane strip 1 that makes use of capillary action lateral flow for performing an immunoassay procedure. The membrane strip 1 comprises of a dilution buffer application pad section 2, a conjugated label pad section 3, a specimen section 4, a membrane section 5, an absorbent pad 6, a specific antigen capture line 7, and a control capture line 8. The dilution buffer application pad section 2 is a segment of the membrane strip 1 where an absorbing pad is mounted and the user will apply a dilution buffer 21. The dilution buffer application pad section 2 is able to pull the dilution buffer 21 onto the membrane strip 1 to start the capillary action. The conjugated label pad section 3 is the next segment of the membrane strip 1 that contains conjugated label solutes 31. The specimen section 4 is a normal section of the membrane strip 1 where the user will apply the specimen that is being tested by the immunoassay procedure of the present invention. The membrane section 5 is a segment of the membrane strip 1 where the dilution buffer 21, the conjugated labels 31, the specimen, the specific antigen capture line 7, and the control capture line 8 interact to determine the results of the immunoassay procedure.

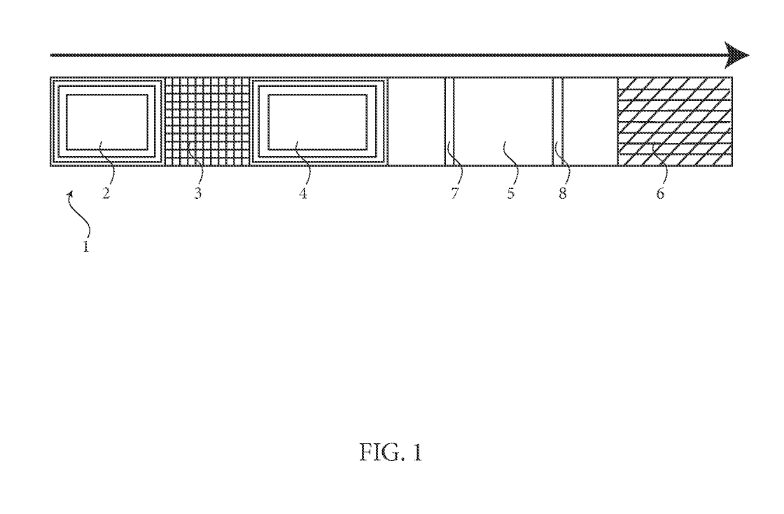

In reference to FIG. 1, the dilution buffer application pad section 2, the conjugated label pad section 3, the specimen section 4, the membrane section 5, and the absorbent pad 6 are connected in linear relationship. The dilution buffer application pad section 2 is adjacently positioned to the conjugated label pad section 3. The conjugated label pad section 3 is adjacently positioned between the dilution buffer application pad section 2 and the specimen section 4. The specimen section 4 is adjacently positioned between the conjugated label pad section 3 and the membrane section 5. The membrane section 5 is adjacently positioned between the specimen section 4 and the absorbent pad 6. The antigen capture line 7 and the control capture line 8 are both positioned on the membrane section 5 spanning the width of the membrane strip 1. The antigen capture line 7 is positioned on the membrane section 5 adjacent to the specimen section 4. The control capture line 8 is positioned on the membrane section 5 adjacent to the absorbent pad 6. The specific antigen capture line 7 contains concentrated amounts of an antigen 71 that corresponds to antibodies 41 that are found in the specimens to be tested. The absorbent pad 6 being positioned at the very end of the membrane strip 1 act as an agent to attract the specimen and a conjugated label solution 51 through capillary action.

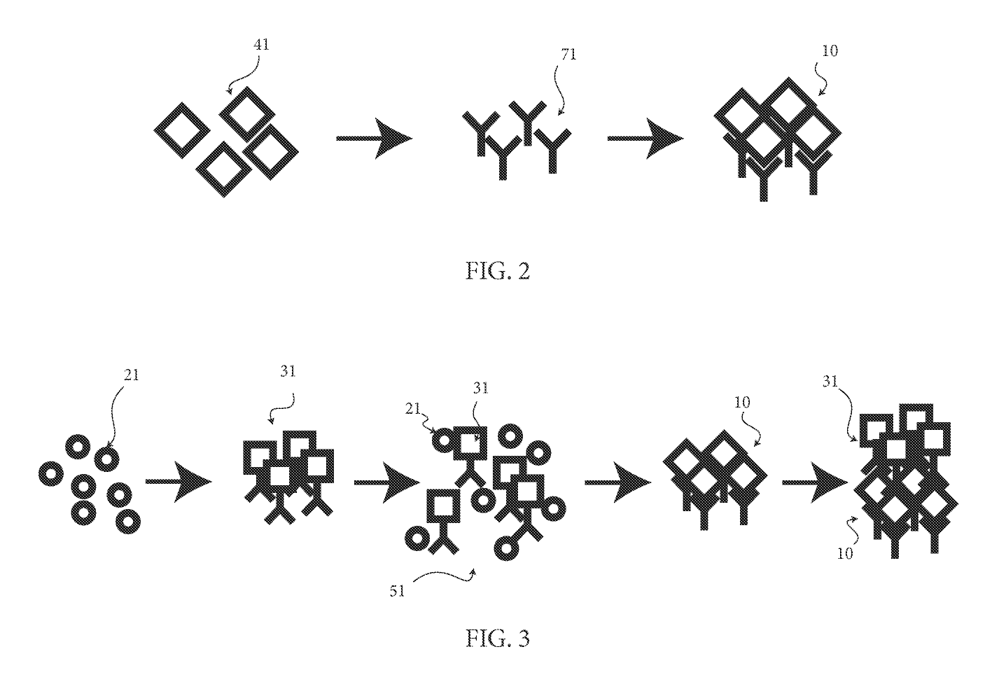

In reference to FIG. 2-3, to use the present invention for a quick immunoassay, the user will proceed with the following steps:

Step 1—Apply specimen directly to the specimen section 4 of the membrane strip 1. The specimen will begin to migrate towards the absorbent pad 6.

Step 2—Apply a dilution buffer 21 to the dilution buffer application pad section 2. The dilution buffer 21 will follow behind the specimen in migrating towards the absorbent pad 6. As the dilution buffer 21 migrates towards the absorbent pad 6, it will first pass by the conjugated label pad section 3. The dilution buffer 21 will solubilize the conjugated label solutes to create the conjugated label solution 51. The dilution buffer 21 becoming the conjugated label solution 51 will continue to migrate toward the absorbent pad 6 by means of capillary action.

Step 3—During the migration of the specimen towards the absorbent pad 6, the specimen will travel through the membrane section 5 and interact with the specific antigen capture line 7. The specimen having antibodies 41 will correspond to the specific antigen in the specific antigen capture line 7. If the antibodies 41 correspond to the antigen 71 in the specific antigen capture line 7, they will bind together and remain in the line with all other molecules continuing to migrate towards the absorbent pad 6. The binding of the antibody 41 and the antigen 71 forms a specific antigen-antibody complex 10. However, if the specific antibodies are not present in the specimen, the specimen will naturally just pass by the specific antigen capture line 7.

Step 4—following the specimen is the conjugated label solution 51 containing the conjugated labels 31. The conjugated labels 31 are specific to the antigen-antibody complex 10. If the antigen-antibody complex 10 is present in the specific antigen capture line 7, the conjugated labels 31 will bind to the antigen-antibody complexes 10. The binding formed by the conjugated labels 31 to the antigen-antibody complexes 10 will activate the conjugated labels 31 to transform the specific antigen capture line 7 into a visible colorimetric line. If the antigen-antibody complex 10 is not present, the conjugated label solution will simply pass by.

Step 5—The conjugated label solution 51 will continue to migrate towards the absorbent pad 6 and encounter the control capture line 8. The control capture line 8 informs the user the validity of the immunoassay procedure. If a specimen was added to the specimen section 4 before the addition of the dilution buffer 21, a colorimetric line will appear in the control capture line 8. If the specimen was not added, a visible colorimetric line will not appear. If the visible colorimetric line does not appear, the control capture line 8 will indicate the invalidity of the test.

Step 6—The conjugated label solution 51 will continue to migrate towards the membrane strip 1 and be captured by the absorbent pad 6.

Unlike traditional lateral flow assays, the present invention has the specimen applied to the membrane strip 1 separately from the dilution buffer 21. With the specimen and the dilution buffer 21 added at different times, this causes the conjugated label solution 51, and the specimen to migrate separately. In the present invention, the specimen will be applied first to migrate through the membrane strip 1 first. The specimen will then be later followed by the dilution buffer 21 and the solubilized conjugate label.

The membrane strip 1 can be made from any sterile material suitable for solutions to move by means of capillary action. The conjugated label solute contained by the conjugated label pad section 3 can be any conjugated label for any specific antigen-antibody complex 10. The specific antigen contained within the specific antigen capture line 7 can be any type of antigen. The type of conjugated label and antibody contained within the conjugated label pad section 3 and the specific antigen capture section is determined by the type of antibody the user is trying to detect in the specimen. The membrane strips 1 can be manufactured to test any specimens for any kinds of antibody.

Although the invention has been explained in relation to its preferred embodiment, it is to be understood that many other possible modifications and variations can be made without departing from the spirit and scope of the invention as hereinafter claimed.

What is claimed is:

1. The apparatus for a lateral-flow rapid test device comprises,
a membrane strip;
the membrane strip comprises of a dilution buffer application pad section, a conjugated label pad section, a specimen section, a membrane section, an absorbent pad, a specific antigen capture line, and a control capture line;
the dilution buffer application pad section, the conjugated label pad section, the specimen section, the membrane section, and the absorbent pad being connected in linear relationship; and
the antigen capture line and the control capture line being positioned on the membrane section.

2. The apparatus for lateral-flow rapid test device as claimed in claim 1 comprises,
the dilution buffer application pad section being adjacently positioned to the conjugated label pad section;
the conjugated label pad section being adjacently positioned between the dilution buffer application pad section and the specimen section;
the specimen section being adjacently positioned between the conjugated label pad section and the membrane section; and
the membrane section being adjacently positioned between the specimen section and the absorbent pad.

3. The apparatus for lateral-flow rapid test device as claimed in claim 1 comprises,
the antigen capture line being positioned on the membrane section adjacent to the specimen section; and
the control capture line being positioned on the membrane section adjacent to the absorbent pad.

4. The method for using the lateral-flow rapid test device comprises,
providing a membrane strip;
the membrane strip comprises a dilution buffer application pad section, a conjugated label pad, a specimen section, a membrane section, an absorbent pad, a specific antigen capture line, and a control capture line;
the dilution buffer application pad section, the conjugated label pad section, the specimen section, the membrane section, and the absorbent pad being connected in linear relationship;
the antigen capture line and the control capture line being positioned on the membrane section; and
the conjugated label pad section having labeled conjugates.

5. The method for using the lateral-flow rapid test device as claimed in claim 4 comprises,
applying a specimen directly to the specimen section;
migrating of the specimen towards the absorbent pad through the membrane section by means of capillary action;
applying a dilution buffer directly to the dilution buffer application pad section;
migrating of the dilution buffer through the membrane strip towards the conjugated label pad section;
solubilizing of the conjugated labels by the dilution buffer to create a labeled conjugate solution; and
migrating of the labeled conjugate solution towards the absorbent pad.

6. The method for using the lateral-flow rapid test device as claimed in claim 5 comprises,
the specific antigen capture line having a specific antigen;
the specimen having antibodies interacts with the specific antigen of the specific antigen capture line;
binding of the antibodies of the specimen to the specific antigen secured to the specific antigen capture line allowing the rest of the materials to continue migrating towards the absorbent pad;
the antigen and the specific antibody binding to create a antibody-antigen complex;
the labeled conjugate solution having the conjugate labels migrating through the specific antigen capture line;
the conjugate labels of the labeled conjugate solution binding to the antigen-antibody complex; and
the binding of the conjugate labels to the antigen-antibody complex transforming the antigen specific capture line into a visible colorimetric line.

7. The method for using the lateral-flow rapid test device as claimed in claim 6 comprises,
the labeled conjugate solution continuing to migrate through the membrane section towards the absorbent pad, wherein the labeled conjugate solution will be captured by the absorbent pad once reached.

8. The method for using the lateral-flow rapid test device as claimed in claim 6 comprises,
the control capture line having a controlled substance;
wherein the controlled substance is activated by the labeled conjugate solution when the specimen is applied to the specimen section; and
activating of the controlled substance to transform the controlled capture line into a controlled visible colorimetric line denoting validity of the experiment.

9. The apparatus for a lateral-flow rapid test device comprises,
a membrane strip;
the membrane strip comprises of a dilution buffer application pad section, a conjugated label pad section, a specimen section, a membrane section, an absorbent pad, a specific antigen capture line, and a control capture line;
the dilution buffer application pad section, the conjugated label pad section, the specimen section, the membrane section, and the absorbent pad being connected in linear relationship;
the antigen capture line and the control capture line being positioned on the membrane section;
the dilution buffer application pad section being adjacently positioned to the conjugated label pad section;
the conjugated label pad section being adjacently positioned between the dilution buffer application pad section and the specimen section;
the specimen section being adjacently positioned between the conjugated label pad section and the membrane section;
the membrane section being adjacently positioned between the specimen section and the absorbent pad;
the antigen capture line being positioned on the membrane section adjacent to the specimen section; and
the control capture line being positioned on the membrane section adjacent to the absorbent pad.
